(19) United States
(12) Patent Application Publication
(10) Pub. No.: US 2003/0032908 A1
(43) Pub. Date: Feb. 13, 2003

(54) Combination day/night splint

(76) Inventor: Terry M. Nayfa, Oklahoma City, OK (US)

Correspondence Address: Crowe & Dunlevy, 1800 Mid-America Tower, 20 North Broadway, Oklahoma City, OK, 73102-8273, US

(21) Appl. No.: 10/213,630

(22) Filed: Aug. 6, 2002

Related U.S. Application Data
(60) Provisional application No. 60/310,320, filed on Aug. 6, 2001.

(51) Int. Cl. A61F 5/052; A61F 5/058

(52) U.S. Cl. 602/27; 602/23

(57) ABSTRACT

The present invention provides an apparatus in which a night splint is incorporated with a leg walker to provide a combination day and night splint. In the presently preferred embodiment, the night splint is removably nested within the leg walker. The outer leg walker may be removed, leaving the night splint in place. This avoids the need to switch between day and night splints.

### RELATED APPLICATION

This application claims priority to Provisional Patent Application No. 60/310,320 filed Aug. 6, 2001.

### FIELD OF THE INVENTION

The present invention relates generally to the area of orthopedic devices and more specifically to supporting devices for the lower leg.

### BACKGROUND OF THE INVENTION

A variety of devices are presently on the market that provide support and stability to injured limbs. Among these devices are those that are designed primarily for day use, and conversely those that are designed primarily for night use.

Devices that are sufficient for day use generally function to enable a person to carry on daily activities that would otherwise be difficult or impossible. Actions such as standing or walking can be made practical by providing support and protection through a lower leg brace. Injury due to many types of accidents and ailments can be alleviated due to use of this type of brace. Additionally, immobilization of the affected area can result in increased rates of healing. A device such as this can allow routine daily tasks while avoiding any impediment to recovery.

Similarly, devices for night use generally function to immobilize a limb or to put the limb in a position that is more conducive to healing than that of the natural position. These devices can serve to hold a foot at a desired angle or may prevent additional injury which may occur during sleep. Night use devices usually are less bulky than their day use counterparts, and typically provide less protection than that required during the day.

Since different devices are used during the day and night periods, a transition between the two devices is necessary. At the end of the applicable period, one of the devices must be removed and replaced with the alternate device. During this transition, the user loses the immobilization, which was present with the attached device. The user must also attach and re-attach the necessary brace, which can take significant time to ensure the brace is properly employed. It is cost effective and practical to utilize a combination day/night device instead of separate day and night devices. These inconveniences and disadvantages create a need for a solution, which reduces risk and inconvenience now present during the transition between the two types of brace.

### BRIEF DESCRIPTION OF THE DRAWINGS

FIG. 1 is a front and side view of a removable leg walker with its adjustable straps in the installed position.

FIG. 2 is a rear and side view of a removable leg walker with its adjustable straps in the installed position.

FIG. 3 is a front and side view of a night splint.

FIG. 4 is a rear and side view of a night splint.

FIG. 5 is a front view of a removable leg walker and a night splint showing the night splint removed from its attachment in the leg walker.

FIG. 6 is a front view of a removable leg walker and a night splint showing the night splint attached to the inside of the leg walker.

### DESCRIPTION

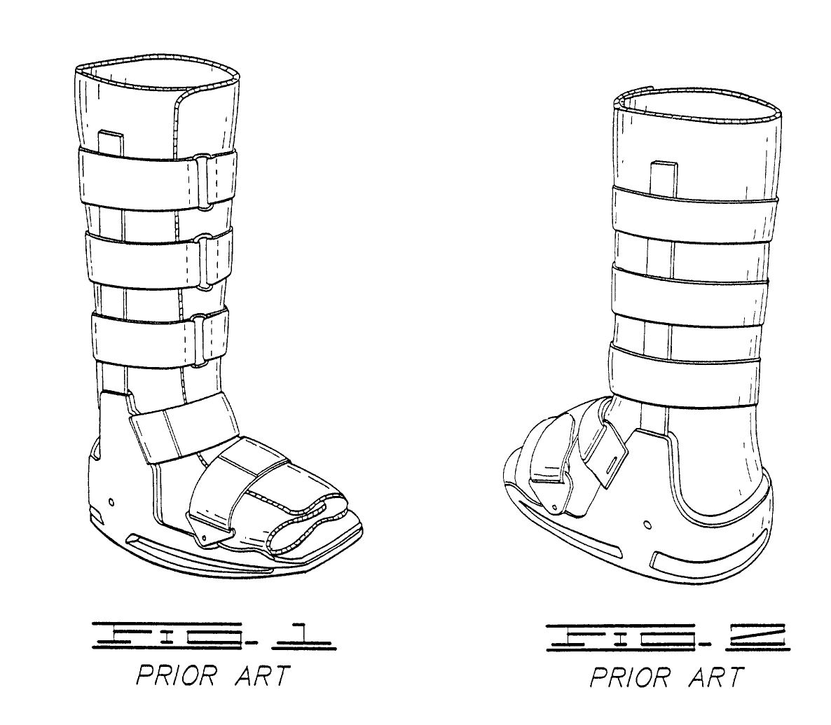

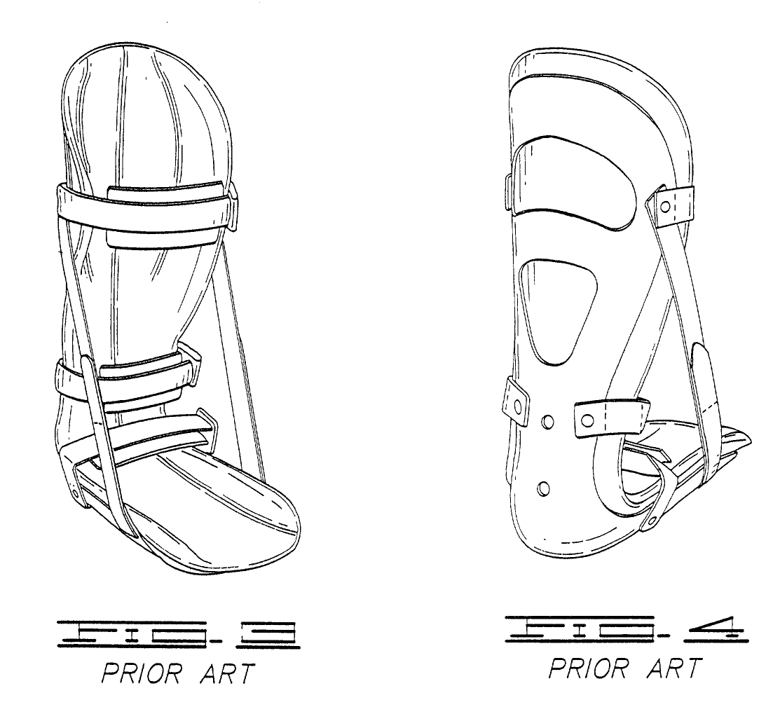

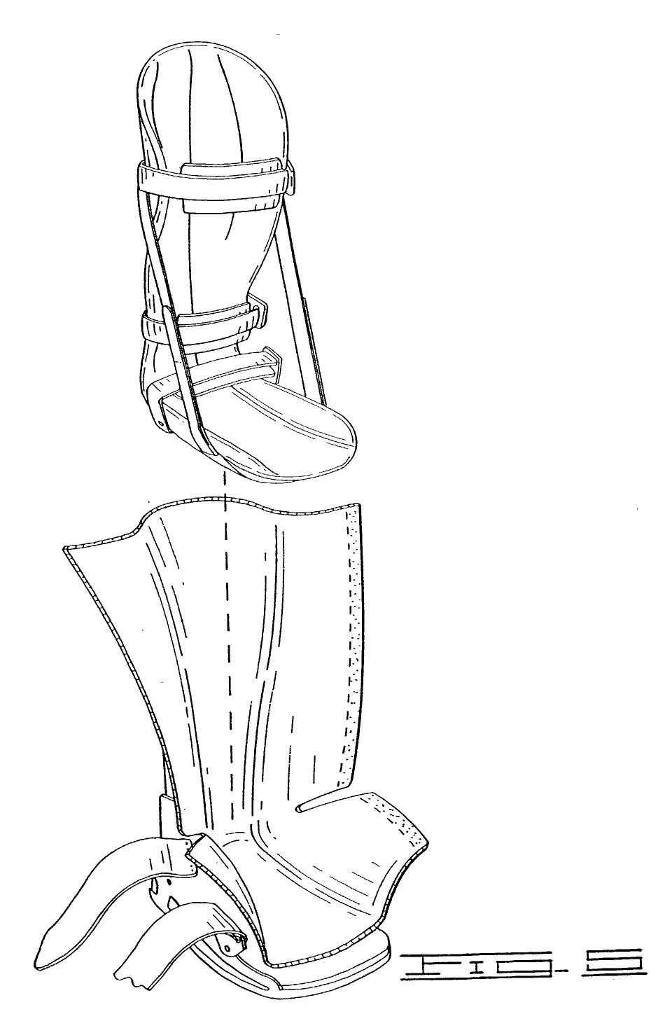

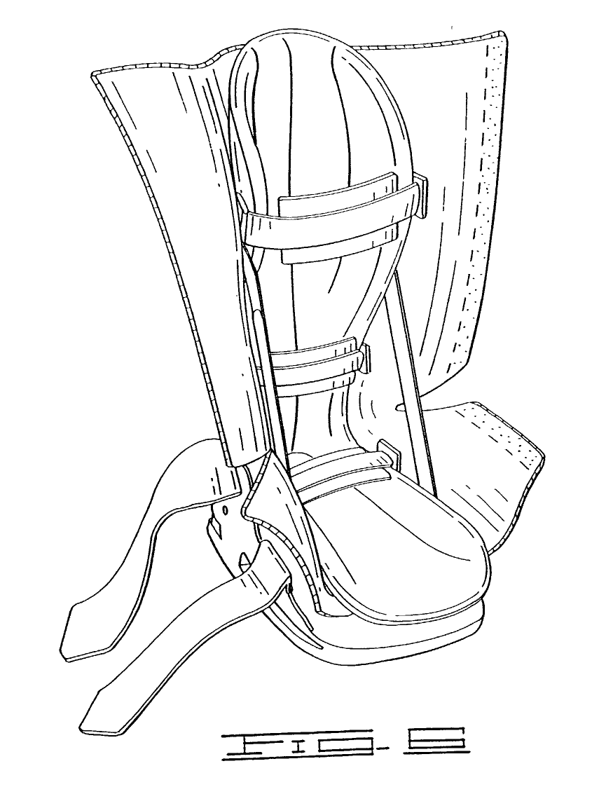

The present invention provides an apparatus 10 in which a night splint 12 (FIG. 3 and FIG. 4) is incorporated with a leg walker 14 (FIG. 1 and FIG. 2). In the presently preferred embodiment, the night splint 12 is removably attached in the leg walker 14 as shown in FIG. 6 (and shown in FIG. 5 in its unattached configuration).

The night splint 12 is utilized by loosening the straps 16 and placing the lower leg into the night splint 12. The straps 16 are then adjusted to the desired point in which comfort and immobilization are concurrently achieved. This configuration is sufficient for use at night.

For periods of desired walking, standing, or other daily activities, the night splint 12 is inserted into the leg walker 14 while the night splint 12 is still attached to the lower leg. Straps 18 are loosened to allow insertion of the night splint 12 into the leg walker 14. These straps 18 are then adjusted to the desired tightness. The entire apparatus 10 may then be worn as required for use during the day. Once daytime use is concluded, the leg walker 14 is simply removed and the night splint 12 is left attached to the lower leg for the desired night use.

It will be clear that the present invention is well adapted to attain the ends and advantages mentioned as well as those inherent therein. While presently preferred embodiments have been described for purposes of disclosure, numerous changes may be made which will readily suggest themselves to those skilled in the art and which are encompassed in the spirit of the invention disclosed.

What is claimed is:

1. A combination day and night splint comprising:
an inner splint portion designed to be worn at all times; and
an outer splint portion, nesting around the inner splint portion, designed to be worn during times of significant walking activity.
